(19) United States
(12) Patent Application Publication
(10) Pub. No.: US 2020/0397609 A1
(43) Pub. Date: Dec. 24, 2020

(54) ARTIFICIAL STOMA DEVICE

(71) Applicant: Yi-Hsin CHANG, Kaohsiung City (TW)

(72) Inventor: Yi-Hsin CHANG, Kaohsiung City (TW)

(21) Appl. No.: 16/450,087

(22) Filed: Jun. 24, 2019

(51) Int. Cl. A61F 5/445; A61F 5/44

(52) U.S. Cl. CPC A61F 5/445; A61F 2005/4455; A61F 5/4407

(57) ABSTRACT

An artificial stoma device includes a hollow first unit and a second unit that are adapted to be respectively inserted into an upstream section and a downstream section of an intestine via an opening of a skin tissue and via a radial side opening of the intestine. The first unit has inlet and outlet portions. The inlet portion has an inlet opening at a distal end thereof that is adapted for facing the upstream section of the intestine. The outlet portion is connected to the inlet portion, and has an outlet opening that is opposite to and in spatial communication with the inlet opening and that is adapted to be exposed from the skin tissue via the opening. The inlet portion, the outlet portion and the second unit are molded as one piece.

### FIELD

The disclosure relates to an artificial organ, and more particularly to an artificial stoma device.

### BACKGROUND

An artificial stoma is an opening of a severed intestine formed as a substitute for excretion when a part of the intestine is cut off due to lesions in the intestine of a patient. In general, an artificial stoma serves as a replacement of a downstream section of the intestine after it is removed, or as a temporary replacement of the downstream section of the intestine as it is recovering.

Referring to FIG. 1, a conventional artificial stoma device 2 is adapted to be installed in a first opening 110 formed in a severed intestine 11 and in a second opening 120 formed in a human body 12. The intestine 11 extends from the inside to the outside of the second opening 120 in such a way that the first opening 110 faces outwardly. The artificial stoma device 2 includes a brace member 21 adapted to be disposed in the first opening 110 of the intestine 11, and a positioning member 22 connected to an outer portion of the brace member 21.

The brace member 21 is hollow and includes an inner ring 211 that is adapted to abut against an inner wall of the intestine 11, an extension tube 212 that is connected to the inner ring 211 and that has a cross-section decreasing in a direction away from the inner ring 211, and an outer ring 213 that is connected to an end of the extension tube 212 opposite to the inner ring 211, and that defines an aperture 219. The positioning member 22 is adapted to be attached to the skin of the human body 12, and includes an annular portion 221 that defines an annular hole 220, and a plate portion 222 that is adapted to be attached to the human body 12. The inner ring 211 has a diameter larger than that of the outer ring 213. When the brace member 21 is disposed inside the intestine 11, a portion 111 of the intestine 11 is expanded by the inner ring 211. The outer ring 213 surrounds the first opening 110 of the intestine 11, and the aperture 219, the intestinal tract of the intestine 11, and the external environment are in spatial communication with each other. The outer ring 213 abuts against the outside of the human body 12, while the inner ring 211 abuts the portion 111 of the intestine 11 against the inner surface of the human body 12, so that the intestine 11 is positioned at the second opening 120. Then, the annular portion 221 of the position member 22 is attached to the outer ring 213 of the brace member 21, while the plate portion 222 is attached to the peripheral area of the second opening 120, so that the brace member 21 is positioned by the positioning member 22. At this point, the aperture 219 is spatially communicated with the space outside the human body 12 via the annular hole 220, and excrement transported by the intestine 11 can be discharged via the aperture 219.

However, besides the abovementioned approach for treating the severed intestine 11, in cases of temporary disposal, a setup in FIG. 2 may be adopted in order to prevent the downstream section of the intestine 11 from becoming necrotic, that is, forming a side opening 119 in the wall of the intestine 11 instead of severing the intestine 11 so that the intestine 11 may continue its normal physiological circulation. When the downstream section of the intestine 11 relative to the side opening 119 has fully recovered, the intestine 11 may be returned to its normal functioning state. However, the artificial stoma device 2 shown in FIG. 1 is not applicable to the setup shown in FIG. 2. If the intestine 11 is directly sutured to the human body 12 such that the side opening is directly connected to the second opening 120 to form an artificial stoma, the excrement may still be transported from the upstream section to the downstream section of the intestine 11 and affect the recovery of the intestine 11. Thus, it is imperative to provide an artificial stoma device that is applicable to the setup shown in FIG. 2.

### SUMMARY

Therefore, the object of the disclosure is to provide an artificial stoma device that can alleviate at least one of the drawbacks of the prior art.

According to the disclosure, the artificial stoma device is adapted to be installed in an abdomen. The abdomen contains an intestine formed with a radial side opening, and has a skin tissue formed with an opening being registered with the radial side opening. The intestine has an upstream section that is disposed upstream of the radial side opening, and a downstream section that is disposed downstream of the radial side opening. The artificial stoma device includes a hollow first unit and a second unit.

The first unit is adapted to be inserted into the upstream section of the intestine via the opening of the skin tissue and the radial side opening of the intestine. The first unit has an inlet portion and an outlet portion. The inlet portion is adapted to be disposed in the upstream section of the intestine, and has an inlet opening at a distal end thereof adapted for facing the upstream section of the intestine. The outlet portion is adapted to be disposed at the opening of the skin tissue and the radial side opening of the intestine, is connected to the inlet portion, and has an outlet opening that is opposite to and in spatial communication with the inlet opening and that is adapted to be exposed from the skin tissue via the opening.

The second unit is adapted to be inserted into the downstream section of the intestine via the opening of the skin tissue and the radial side opening of the intestine. The inlet portion, the outlet portion and the second unit are molded as one piece.

### BRIEF DESCRIPTION OF THE DRAWINGS

Other features and advantages of the disclosure will become apparent in the following detailed description of the embodiments with reference to the accompanying drawings, of which:

FIG. 1 is a fragmentary sectional view illustrating a conventional artificial stoma device connecting an intestine to a human abdomen;

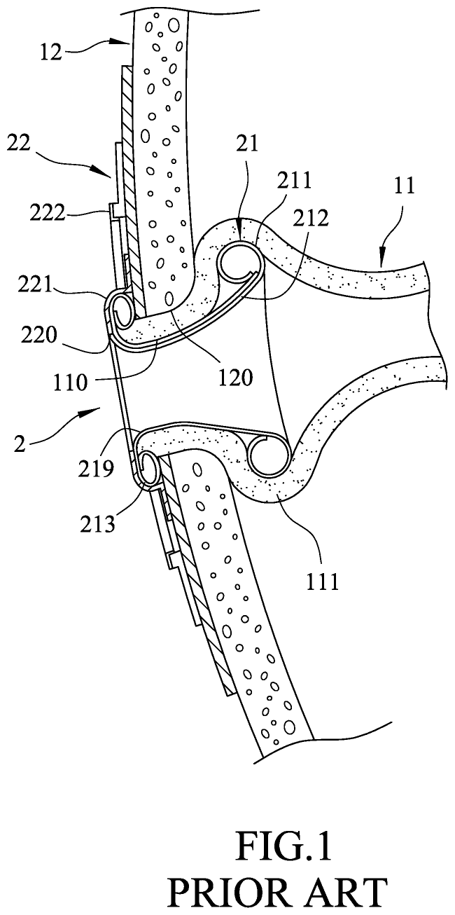

FIG. 2 is a fragmentary sectional view illustrating a setup of forming a side opening in the intestine;

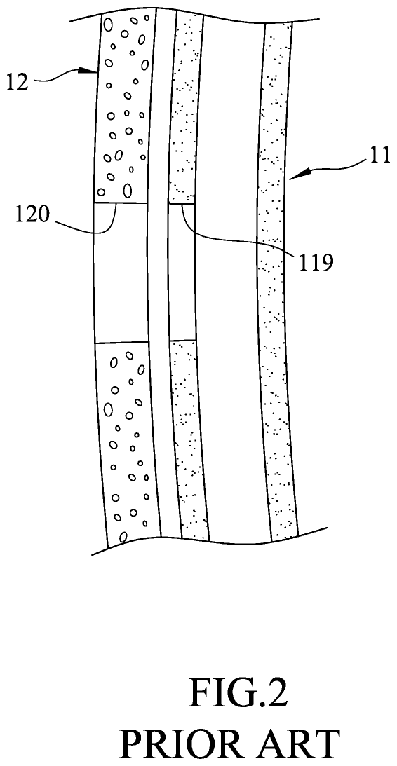

FIG. 3 is an exploded sectional view of a first embodiment of the artificial stoma device according to the disclosure;

FIG. 4 is a fragmentary sectional view illustrating the installation process of the first embodiment in an abdomen;

FIG. 5 is an exploded sectional view of a second embodiment of the artificial stoma device according to the disclosure;

FIG. 6 is an exploded sectional view of a third embodiment of the artificial stoma device according to the disclosure; and

FIG. 7 is an exploded sectional view of a fourth embodiment of the artificial stoma device according to the disclosure.

### DETAILED DESCRIPTION

Before the present invention is described in greater detail, it should be noted that where considered appropriate, reference numerals or terminal portions of reference numerals have been repeated among the figures to indicate corresponding or analogous elements, which may optionally have similar characteristics.

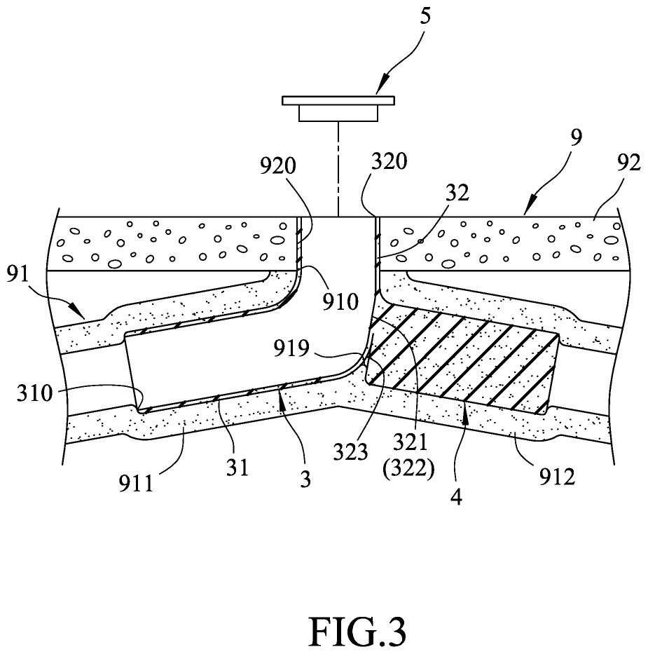

Referring to FIG. 3, a first embodiment of an artificial stoma device according to the present disclosure is adapted to be installed in an abdomen 9 of a patient. The abdomen 9 contains an intestine 91 formed with a radial side opening 910, and has a skin tissue 92 formed with an opening 920 which is registered with the radial side opening 910. The intestine 91 has an upstream section 911 that is disposed upstream of the radial side opening 910, and a downstream section 912 that is disposed downstream of the radial side opening 910. The first embodiment includes a first unit 3 adapted to be inserted into the upstream section 911 of the intestine 91 via the opening 920 of the skin tissue 92 and the radial side opening 910 of the intestine 91, a second unit 4 adapted to be inserted into the downstream section 912 of the intestine 91 via the opening 920 of the skin tissue 92 and the radial side opening 910 of the intestine 91, and a cover unit 5.

The first unit 3 is hollow, and has an inlet portion 31 and an outlet portion 32. The inlet portion 31 is adapted to be disposed in the upstream section 911 of the intestine 91, and has an inlet opening 310 at a distal end thereof that is adapted for facing the upstream section 911 of the intestine 91.

The outlet portion 32 is adapted to be disposed at the opening 920 of the skin tissue 92 and the radial side opening 910 of the intestine 91, is connected to the inlet portion 31, and has an outlet opening 320 and a connecting segment 321. The outlet opening 320 is opposite to and in spatial communication with the inlet opening 310 and is adapted to be exposed from the skin tissue 92 via the opening 920. The connecting segment 321 is adapted to be adjacent to the downstream section 912 of the intestine 91, is in contact with the second unit 4, and has a linking section 322 that is in contact with the second unit 4 and a clamping section 323 that extends from the linking section 322. An acute angle is formed between the clamping section 323 and the second unit 4. The clamping section 323 is adapted to cooperate with the second unit 4 for clamping a portion 919 of the intestine 91 therebetween.

The second unit 4 is solid, and the inlet and outlet portions 31, 32 of the first unit 3 and the second unit 4 are molded as one piece.

The cover unit 5 is detachably coupled to the outlet portion 32 of the first unit 3 and is adapted for blocking the opening 920 of the skin tissue 92.

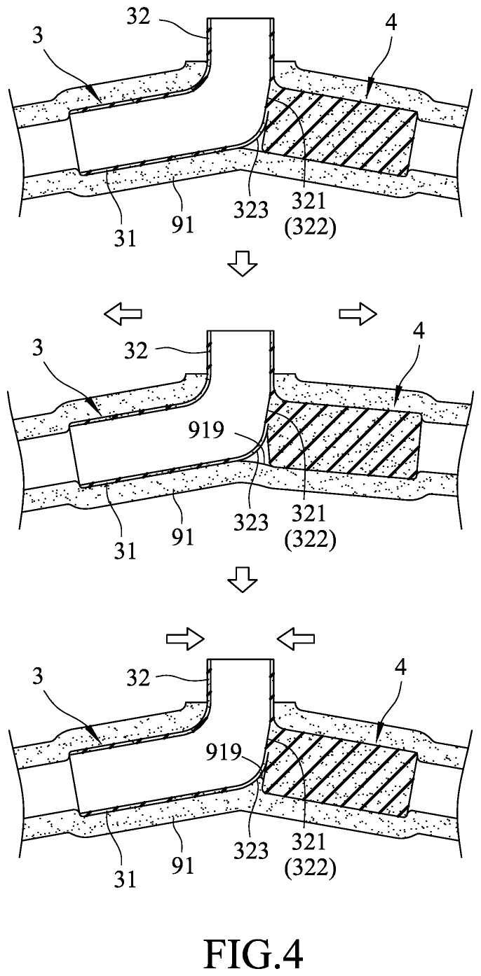

Referring to FIG. 4 in company with FIG. 3, to install the artificial stoma device of the present embodiment into the abdomen 9 of the patient, the first and second units 3, 4 are respectively inserted into the upstream and downstream sections 911, 912 of the intestine 91. Then, the clamping section 323 of the first unit 3 cooperates with the second unit 4 to clamp the portion 919 of the intestine 91 therebetween for positioning the first and second units 3, 4 in the abdomen 9.

More specifically, in the present embodiment, the first and second units 3, 4 are made of elastic materials, such as biocompatible medical grade silicones, to avoid discomfort for the patient. Thus, a distance between the clamping section 323 of the first unit 3 and the second unit 4 can be increased by an external force exerted onto the artificial stoma device. When the artificial stoma device is released, the clamping section 323 and the second unit 4 will restore to clamp the portion 919 of the intestine 91 therebetween.

After installation of the present embodiment is completed, the upstream section 911 of the intestinal 91, the inlet and outlet openings 310, 320 of the first unit 3, and the opening 920 of the skin tissue 92 are all in spatial communication such that an artificial stoma is formed. Thus, excrement transported through the upstream section 911 of the intestinal 91 may be guided into the first unit 3 of the artificial stoma device via the inlet opening 310 of the inlet portion 31, and then be discharged from the first unit 3 via the outlet opening 320 of the outlet portion 32. Also, in virtue of the second unit 4, no excrement is allowed to pass through the downstream section 912 of the intestinal 91 so that the downstream section 912 is allowed to rest for the time being.

It should be noted that, the cover unit 5 may be a piece of gauze, or any object that is sterilized and that has a shielding effect. The cover unit 5 protects the intestine 91 from direct exposure to external environment, and minimizes a risk of infection.

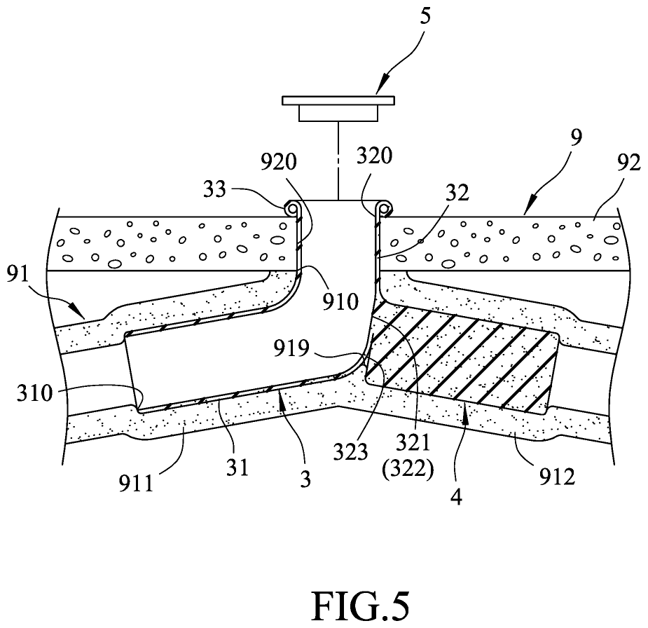

Referring to FIG. 5, a second embodiment of the present disclosure is similar to the first embodiment, and a difference therebetween lies in that the first unit 3 of the second embodiment further includes a positioning member 33.

The positioning member 33 has an end connected to the outlet portion 32 and adjacent to the outlet opening 320, and an opposite end adapted to be disposed at a periphery of the opening 920 and secured to the skin tissue 92.

In virtue of the positioning member 33, the artificial stoma device of the second embodiment is more securely installed in the abdomen 9, and the cover unit 5 may be coupled to the positioning member 33 to provide the same shielding effect as in the previous embodiment.

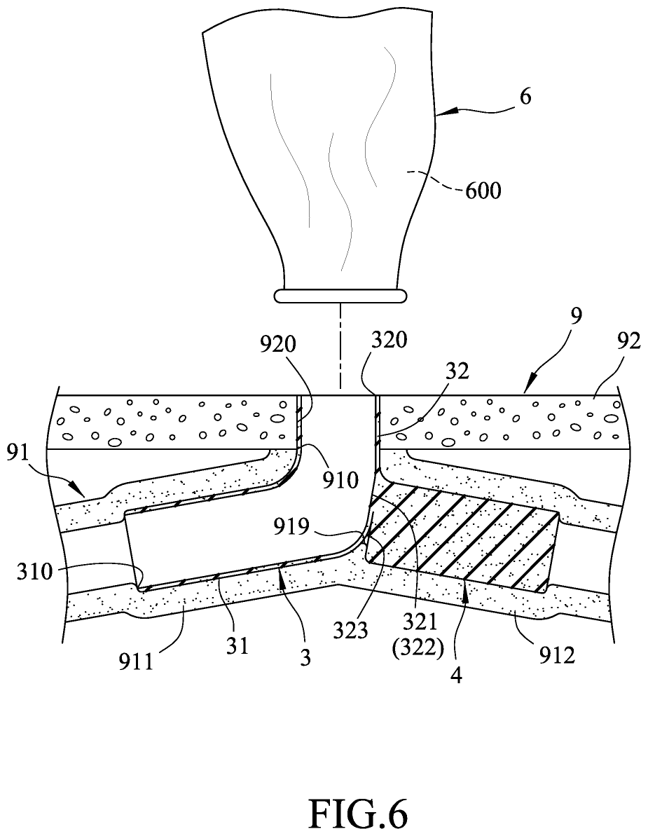

Referring to FIG. 6, a third embodiment of the present disclosure is similar to the first embodiment, and a difference therebetween lies in that the third embodiment further includes a receiving unit 6.

The receiving unit 6 is detachably coupled to the outlet portion 32 of the first unit 3, and defines a receiving space 600 that is adapted to be outside of the skin tissue 92 and that is in spatial communication with the outlet opening 320 of the outlet portion 32.

In such a manner, the receiving space 600 of the receiving unit 6 is able to collect and temporally store the excrement discharged from the opening 920 of the skin tissue 92. Once the excrement reaches a certain amount, the receiving unit 6 may be detached and replaced to maintain hygiene and minimize the risk of infection.

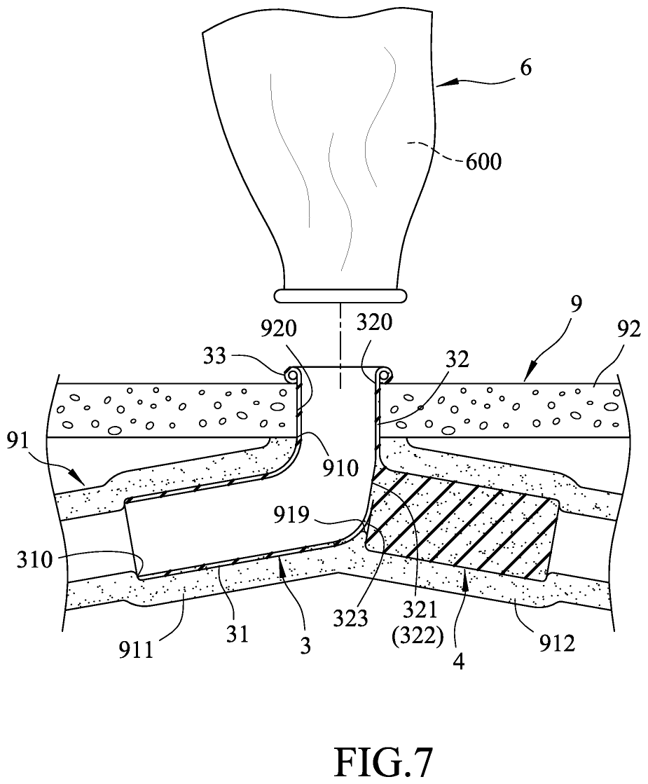

Referring to FIG. 7, a fourth embodiment of the present disclosure is similar to the third embodiment, and a difference therebetween lies in that the first unit 3 of the fourth embodiment further includes a positioning member 33.

The positioning member 33 has an end connected to the outlet portion 32 of the first 3 and adjacent to the outlet opening 320 of the outlet portion 32, and an opposite end adapted to be disposed at a periphery of the opening 920 and secured to the skin tissue 92. The receiving unit 6 is detachably coupled to the positioning member 33 of the first unit 3.

Similar to the second embodiment, the installation of the artificial stoma device to the abdomen 9 is strengthened in virtue of the positioning member 33. Moreover, the receiving unit 6 provides the same effect for maintaining hygiene and minimizing the risk of infection as it does in the third embodiment.

In the description above, for the purposes of explanation, numerous specific details have been set forth in order to provide a thorough understanding of the embodiments. It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. An artificial stoma device adapted to be installed in an abdomen, the abdomen containing an intestine which is formed with a radial side opening, and having a skin tissue which is formed with an opening being registered with the radial side opening, the intestine having an upstream section that is disposed upstream of the radial side opening and a downstream section that is disposed downstream of the radial side opening, said artificial stoma device comprising:
a hollow first unit adapted to be inserted into the upstream section of the intestine via the opening of the skin tissue and the radial side opening of the intestine, said first unit having
an inlet portion that is adapted to be disposed in the upstream section of the intestine, and that has an inlet opening at a distal end thereof adapted for facing the upstream section of the intestine, and
an outlet portion that is adapted to be disposed at the opening of the skin tissue and the radial side opening of the intestine, that is connected to the inlet portion, and that has an outlet opening being opposite to and in spatial communication with said inlet opening and adapted to be exposed from the skin tissue via the opening; and
a second unit adapted to be inserted into the downstream section of the intestine via the opening of the skin tissue and the radial side opening of the intestine, said inlet portion, said cutlet portion and said second unit being molded as one piece.

2. The artificial stoma device as claimed in claim 1, wherein said outlet portion of said first unit has a connecting segment that is adapted to be adjacent to the downstream section of the intestine and that is in contact with said second unit.

3. The artificial stoma device as claimed in claim 2, wherein said connecting segment has:
a linking section that is in contact with said second unit; and
a clamping section that cooperates with said second unit for clamping a portion of the intestine therebetween.

4. The artificial stoma device as claimed in claim wherein said first unit further has a positioning member that has an end connected to said outlet portion and being adjacent to said outlet opening, and an opposite end adapted to be disposed at a periphery of the opening and secured to the skin tissue.

5. The artificial stoma device as claimed in claim 4 further comprising a cover unit detachably coupled to said outlet portion of said first unit and adapted for blocking the opening of the skin tissue.

6. The artificial stoma device as claimed in claim 4, further comprising a receiving unit detachably coupled to said outlet portion of said first unit and defining a receiving space that is adapted to be outside of the skin tissue and that is in spatial communication with said outlet opening.

7. The artificial stoma device as claimed in claim 1, further comprising a cover unit detachably coupled to said positioning member of said first unit and adapted for blocking the opening of the skin tissue.

8. The artificial stoma device as claimed in claim I, further comprising a receiving unit detachably coupled to said outlet portion of said first unit and defining a receiving space that is adapted to be outside of the skin tissue and that is in spatial communication with said outlet opening.

9. The artificial stoma device as claimed in claim wherein said second unit is solid.

10. The artificial stoma device as claimed in claim 2, wherein said second unit is solid.

11. The artificial stoma device as claimed in claim 3, wherein said second unit is solid.

12. The artificial stoma device as claimed in claim 4, wherein said second unit is solid.

13. The artificial stoma device as claimed in claim 5, wherein said second unit is solid.

14. The artificial stoma device as claimed in claim 6, wherein said second unit is solid.

15. The artificial stoma device as claimed in claim 7, wherein said second unit is solid.

16. The artificial stoma device as claimed in claim 8, wherein said second unit is solid.
